(19) United States
(12) Patent Application Publication
(10) Pub. No.: US 2013/0172970 A1
(43) Pub. Date: Jul. 4, 2013

(54) Heat Patch For Obesity

(71) Applicant: Tuming You, Xiamen (CN)

(72) Inventor: Tuming You, Xiamen (CN)

(21) Appl. No.: 13/533,941

(22) Filed: Jun. 26, 2012

Related U.S. Application Data
(63) Continuation-in-part of application No. 13/471,138, filed on May 14, 2012.
Application No. 13/471,138 is a continuation of application No. 13/341,878, filed on Dec. 30, 2011.

(51) Int. Cl. A61K 33/24; A61P 3/04; A61K 35/12; A61K 9/70; A61F 7/00; A61F 7/03

(52) U.S. Cl. 607/114; 607/96; 424/617; 424/520; 424/443

(57) ABSTRACT

Provided in the present invention are a composition for ameliorating or treating obesity and methods of making and using the same.

### CROSS-REFERENCE TO RELATED APPLICATIONS

This application is a continuation-in-part of U.S. application Ser. No. 13/471,138, filed on May 14, 2012. This application is also a continuation-in-part of U.S. application Ser. No. 13/341,878, filed on Dec. 30, 2011. The teachings in these applications are incorporated herein in their entirety by reference.

### FIELD OF THE INVENTION

The invention is related to methods and articles for ameliorating or treating obesity.

### BACKGROUND OF THE INVENTION

Obesity is a medical condition in which excess body fat has accumulated to the extent that it may have an adverse effect on health, leading to reduced life expectancy and/or increased health problems (WHO 2000 p. 6; Haslam D W, James W P (2005). “Obesity”. Lancet 366 (9492): 1197-209). Body mass index (BMI), a measurement which compares weight and height, defines people as overweight (pre-obese) if their BMI is between 25 and 30 kg/m², and obese when it is greater than 30 kg/m² (WHO 2000 p. 9)

Obesity increases the likelihood of various diseases, particularly heart disease, type 2 diabetes, obstructive sleep apnea, certain types of cancer, and osteoarthritis (Haslam et al., 2005). Obesity is most commonly caused by a combination of excessive food energy intake, lack of physical activity, and genetic susceptibility, although a few cases are caused primarily by genes, endocrine disorders, medications or psychiatric illness. Evidence to support the view that some obese people eat little yet gain weight due to a slow metabolism is limited; on average obese people have a greater energy expenditure than their thin counterparts due to the energy required to maintain an increased body mass (Adams J P, Murphy P G (July 2000). “Obesity in anaesthesia and intensive care”. Br J Anaesth 85 (1): 91-108).

Obesity is a leading preventable cause of death worldwide, with increasing prevalence in adults and children, and authorities view it as one of the most serious public health problems of the 21st century (Barness L A, Opitz J M, Gilbert-Barness E (December 2007). “Obesity: genetic, molecular, and environmental aspects”. Am. J. Med. Genet. A 143A (24): 3016-34). Obesity is stigmatized in much of the modern world (particularly in the Western world), though it was widely perceived as a symbol of wealth and fertility at other times in history, and still is in some parts of the world (Haslam et al., 2005; Woodhouse R (2008). “Obesity in art: A brief overview”. Front Horm Res. Frontiers of Hormone Research 36: 271-86).

According to traditional Chinese medicine, blocking of the free flow of human biological system happens in certain places (points) in the human body and cause diseases including obesity to occur. A particular disorder corresponds to blocking of a particular point or set of points in the biological system. Stimulating one of these points can free up the flow of biological system of human being so as to treat or ameliorate a disease arising out of blocking of such point or set of points. According to this school of medicine, a method of therapeutics—acupuncture—was developed and practiced in China for thousands of years. The oldest medical book known, written in China 4000 years ago, describes the use of acupuncture to treat medical problems. The use of the treatment spread to other Asian countries and to other regions of the world, including to Europe by the 1700s. In the United States, acupuncture has been used for about 200 years.

Research on acupuncture began in the United States in 1976. Twenty years later, according to National Cancer Institute, the US Food and Drug Administration (FDA) approved the acupuncture needle as a medical device. Many illnesses are treated with acupuncture, but it is used mainly to control pain, including pain in cancer patients and to help control nausea and vomiting. Its primary use in cancer patients has been as an addition to conventional (standard) therapy.

The FDA approved acupuncture needles for use by licensed practitioners in 1996. The FDA requires that sterile, nontoxic needles be used and that they be labeled for single use by qualified practitioners only.

Acupuncture has been used in obesity management. Numerous reported studies have shown that acupuncture is effective for obesity (see a review article by Lacey, et al., “Acupuncture for the treatment of obesity: a review of the evidence” International Journal of Obesity (2003) 27, 419-427)

A common side effect of acupuncture is concern of infection caused by cross-contamination of acupuncture needles that may include viral or bacterial pathogens. Other concerns may arise out of patient's negative reactions against piercing of acupuncture needles or misapplication of such needles by a practitioner. Further, access to acupuncture by a practitioner is limited so as to limit the effect of acupuncture.

Therefore, there is a need for a different way of acupuncture treating or management paradigm for obesity.

The embodiments described below address the above identified needs and issues.

### SUMMARY OF THE INVENTION

In one aspect of the present invention, it is provided a composition for obesity, the composition comprises a heat generation component, the composition is formulated in a formulation for selective application to a set of acupoints or one or more acupoint(s) for obesity (“acupoint(s) for obesity”) in a patient, and the composition generates heat upon application to the acupoint(s) for obesity for a sufficient period of time to generate an effect to ameliorate or treat obesity.

In some embodiments of the composition, the set of acupoints comprises shangwan, zhongwan, shenque, shenshu, taixi, and laogong acupoints of a user.

In some embodiments of the composition, optionally in combination with any or all of the above various embodiments, the composition is capable of generating heat for at least 1 hour (e.g., 1 hour, 2 hours, 4 hours, 8 hours, or 10 hours) after application to the set of acupoints or one or more acupoint(s) for obesity.

In some embodiments of the composition, optionally in combination with any or all of the above various embodiments, the heat generation component comprises a rare earth component.

In some embodiments of the composition, optionally in combination with any or all of the above various embodiments, the heat generation component comprises a rare earth magnetic powder, saussurea in volucrata, musk, sea otter, chuanwu, caowu, lilac, menthol, meiyao, wujiapi, and maqianzhi.

In some embodiments of the composition, optionally in combination with any or all of the above various embodiments, the formulation is a patch comprising a cover. In some embodiments, the cover is a plastic, rubber, or paper cover.

In another aspect of the present invention, it is provided a method of fabricating a composition for obesity, comprising providing a heat generation component, and formulating the composition in a formulation for selective application to a set of acupoints or one or more acupoint(s) for obesity (“acupoint(s) for obesity”) in a patient. The composition generates heat upon application to the set of acupoints or one or more acupoint(s) for obesity for a sufficient period of time to generate an effect to ameliorate or treat obesity.

In some embodiments of the method, the set of acupoints comprises the shangwan, zhongwan, shenque, and Zusanli acupoints of a user.

In some embodiments of method, optionally in combination with any or all of the above various embodiments, the composition is capable of generating heat for at least 1 hour (e.g., 1 hour, 2 hours, 4 hours, 8 hours, or 10 hours) after application to the set of acupoints or one or more acupoint(s) for obesity.

In some embodiments of method, optionally in combination with any or all of the above various embodiments, the heat generation component comprises a rare earth component.

In some embodiments of method, optionally in combination with any or all of the above various embodiments, the heat generation component comprises a rare earth magnetic powder, saussurea in volucrata, musk, sea otter, chuanwu, caowu, lilac, menthol, meiyao, wujiapi, and maqianzhi.

In some embodiments of method, optionally in combination with any or all of the above various embodiments, the formulation is a patch comprising a cover. In some embodiments, the cover is a plastic, rubber, or paper cover.

In a further embodiment of the present invention, it is provided a method of ameliorating or treating obesity, comprising applying a composition at least once per day to a set of acupoints or one or more acupoint(s) for obesity (“acupoint(s) for obesity”) in a user, which composition comprising a heat generation component, wherein the composition is formulated in a formulation for selective application to the set of acupoints or one or more acupoint(s) for obesity over the entire length of a prescribed treatment course, wherein the composition generates heat upon application to the set of acupoints or one or more acupoint(s) for obesity for a sufficient period of time to generate an effect to ameliorate obesity.

In some embodiments of the method, the set of acupoints comprises the shangwan, zhongwan, shenque, and Zusanli acupoints of a user.

In some embodiments of method, optionally in combination with any or all of the above various embodiments, the composition is capable of generating heat for at least 1 hour (e.g., 1 hour, 2 hours, 4 hours, 8 hours, or 10 hours) after application to the set of acupoints or one or more acupoint(s) for obesity.

In some embodiments of method, optionally in combination with any or all of the above various embodiments, the heat generation component comprises a rare earth component.

In some embodiments of method, optionally in combination with any or all of the above various embodiments, the heat generation component comprises a rare earth magnetic powder, saussurea in volucrata, musk, sea otter, chuanwu, caowu, lilac, menthol, meiyao, wujiapi, and maqianzhi.

In some embodiments of method, optionally in combination with any or all of the above various embodiments, the formulation is a patch comprising a cover. In some embodiments of method, the cover is a plastic, rubber, or paper cover.

In some embodiments of method, optionally in combination with any or all of the above various embodiments, the treatment course lasts for about 1 month, 3 months, 6 months, or 12 months.

### BRIEF DESCRIPTION OF THE DRAWINGS

FIGS. 1A and 1B show acupoints for obesity as embodiments of the present invention.

### DESCRIPTION OF EXEMPLARY EMBODIMENTS OF THE INVENTION

In one aspect of the present invention, it is provided a composition for obesity, the composition comprises a heat generation component, the composition is formulated in a formulation for selective application to a set of acupoints or one or more acupoint(s) for obesity (“acupoint(s) for obesity”) in a patient, and the composition generates heat upon application to the acupoint(s) for obesity for a sufficient period of time to generate an effect to ameliorate or treat obesity. As used herein, the term obesity includes the disorder as diagnosed by general medical standards and symptoms of obesity.

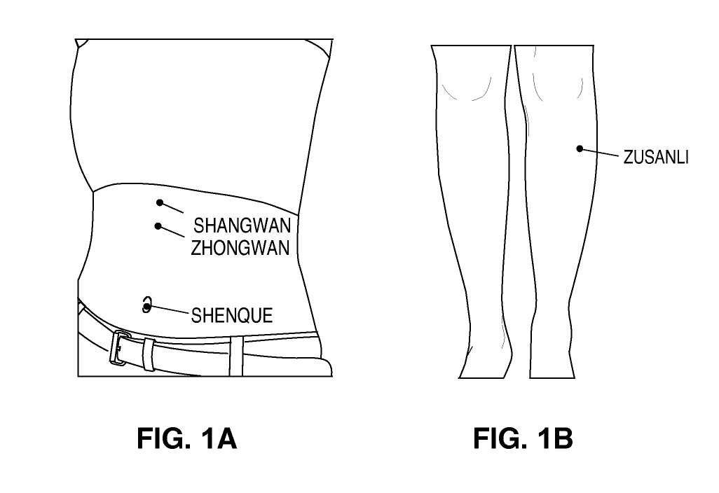

The set of acupoints for obesity comprises the shangwan, zhongwan, shenque, and Zusanli acupoints of a user (FIGS. 1A-1B).

In some embodiments, optionally in combination with any or all of the above various embodiments, the composition is capable of generating heat for at least 10 minutes, e.g., 20 minutes, 30 minutes, 60 minutes (1 hour), 2 hours, 3 hours, 4 hours, 5 hours, 6 hours, 7 hours, 8 hours, 10 hours, 12 hours, or 24 hrs. In some embodiments, the composition is capable of generating heat for at least 2 hours after application to the acupoint(s) for obesity.

In some embodiments of the composition, optionally in combination with any or all of the above various embodiments, the heat generation component comprises a rare earth component. Such rare earth component can be a rare earth magnetic powder (e.g., samarium-cobalt magnetic material or neodymium based magnetic material) or a rare earth oxide, which can be any oxide of a rare earth element. Rare earth elements include lanthanide series elements and actinide series elements. Lanthanide series elements are Lanthanum, Cerium, Praseodymium, Neodymium, Promethium, Samarium, Europium, Gadolinium, Terbium, Dysprosium, Holmium, Erbium, Thulium, Ytterbium, and Lutetium; Actinide series elements are Actinium, Thorium, Protactinium, Uranium, Neptunium, Plutonium, Americium, Curium, Berkelium, Californium, Einsteinium, Fermium, Mendelevium, Nobelium, and Lawrencium.

In some embodiments of the composition, optionally in combination with any or all of the above various embodiments, the heat generation component comprises a rare earth magnetic powder, saussurea in volucrata, musk, sea otter, chuanwu, caowu, lilac, menthol, meiyao, wujiapi, and maqianzhi.

In some embodiments of the composition, optionally in combination with any or all of the above various embodiments, the formulation is a patch comprising a cover. In some embodiments, the cover can be e.g., a plastic, rubber, or paper cover.

In another aspect of the present invention, it is provided a method of fabricating a composition for obesity, comprising providing a heat generation component, and formulating the composition in a formulation for selective application to a set of acupoints or one or more acupoint(s) for obesity (“acupoint(s) for obesity”) in a patient. The composition generates heat upon application to the set of acupoints or one or more acupoint(s) for obesity for a sufficient period of time to generate an effect to ameliorate or treat obesity.

In some embodiments of the composition, the set of acupoints comprises the shangwan, zhongwan, shenque, and Zusanli acupoints of a user.

In some embodiments of the method, optionally in combination with any or all of the above various embodiments, the composition is capable of generating heat for at least 10 minutes, e.g., 20 minutes, 30 minutes, 60 minutes (1 hour), 2 hours, 3 hours, 4 hours, 5 hours, 6 hours, 7 hours, 8 hours, 10 hours, 12 hours, 14 hours, or 24 hours. In some embodiments, the composition is capable of generating heat for at least 2 hours after application to the acupoint(s) for obesity.

In some embodiments of the method, optionally in combination with any or all of the above various embodiments, the heat generation component comprises a rare earth component. Such rare earth component can be a rare earth magnetic powder (e.g., samarium-cobalt magnetic material or neodymium based magnetic material) or a rare earth oxide, which can be any oxide of a rare earth element. Rare earth elements include lanthanide series elements and actinide series elements. Lanthanide series elements are Lanthanum, Cerium, Praseodymium, Neodymium, Promethium, Samarium, Europium, Gadolinium, Terbium, Dysprosium, Holmium, Erbium, Thulium, Ytterbium, and Lutetium; Actinide series elements are Actinium, Thorium, Protactinium, Uranium, Neptunium, Plutonium, Americium, Curium, Berkelium, Californium, Einsteinium, Fermium, Mendelevium, Nobelium, and Lawrencium.

In some embodiments of the method, optionally in combination with any or all of the above various embodiments, the formulation is a patch comprising a cover. In some embodiments, the cover can be e.g., a plastic, rubber, or paper cover.

In a further aspect of the present invention, it is provided a method of ameliorating or treating obesity, comprising applying a composition at least once per day to a set of acupoints or one or more acupoint(s) for obesity (“acupoint(s) for obesity”) in a user, which composition comprising a heat generation component, wherein the composition is formulated in a formulation for selective application to the set of acupoints or one or more acupoint(s) for obesity over the entire length of a prescribed treatment course, wherein the composition generates heat upon application to the set of acupoints or one or more acupoint(s) for obesity for a sufficient period of time to generate an effect to ameliorate obesity.

In some embodiments of the method, the composition is capable of generating heat for at least 10 minutes, e.g., 20 minutes, 30 minutes, 60 minutes (1 hour), 2 hours, 3 hours, 4 hours, 5 hours, 6 hours, 7 hours, 8 hours, 10 hours, 12 hours, 15 hours or a day. In some embodiments, the composition is capable of generating heat for at least 2 hours after application to the acupoint(s) for obesity. In some embodiments, the composition is capable of generating heat for at least 2 hours after application to the acupoint(s) for obesity.

In some embodiments of the method, optionally in combination with any or all of the above various embodiments, the heat generation component comprises a rare earth component. Such rare earth component can be a rare earth magnetic powder (e.g., samarium-cobalt magnetic material or neodymium based magnetic material) or a rare earth oxide, which can be any oxide of a rare earth element. Rare earth elements include lanthanide series elements and actinide series elements. Lanthanide series elements are Lanthanum, Cerium, Praseodymium, Neodymium, Promethium, Samarium, Europium, Gadolinium, Terbium, Dysprosium, Holmium, Erbium, Thulium, Ytterbium, and Lutetium; Actinide series elements are Actinium, Thorium, Protactinium, Uranium, Neptunium, Plutonium, Americium, Curium, Berkelium, Californium, Einsteinium, Fermium, Mendelevium, Nobelium, and Lawrencium.

In some embodiments of the method, optionally in combination with any or all of the above various embodiments, the heat generation component comprises a rare earth magnetic powder, saussurea in volucrata, musk, sea otter, chuanwu, caowu, lilac, menthol, meiyao, wujiapi, and maqianzhi.

In some embodiments of method, optionally in combination with any or all of the above various embodiments, the formulation is a patch comprising a cover. In some embodiments of method, the cover is a plastic, rubber, or paper cover.

In some embodiments of method, optionally in combination with any or all of the above various embodiments, the treatment course lasts for about 1 month, 3 months, 6 months, or 12 months.

Weight loss of a user of the present invention can be readily achieved. For example, weight loss of about 1 kilogram, 2 kilograms, 5 kilograms, 7 kilograms, 10 kilograms or above can be readily achieved over the course of at least one course of treatment. One possible mechanism leading to the weight loss is food intake reduction of a user relative to the user's normal amount of food intake. For example, a person who practices the method of invention can achieve food intake reduction by about 5%, 10%, 20%, 30%, 40%, or about 50%. Food intake is a critical factor leading to obesity. Any reduction of food intake would be technologically significant in weight management.

As used herein, the term “heat generation component” can be any component capable generating heat upon exposure to moisture or air that provides a local temperature above body temperature (e.g., 37° C.) to about 50° C. Local temperature refers to the temperature of the skin area in contact with a composition disclosed herein. Such heat generation can last at least about 10 minutes, e.g., e.g., 20 minutes, 30 minutes, 60 minutes (1 hour), 2 hours, 3 hours, 4 hours, 5 hours, 6 hours, 7 hours, 8 hours, 10 hours, 12 hours, 14 hours, or 24 hours. An example of the heat generating component is a chemical composition commonly in baby products or winter comfort products. For example, in some embodiments, the heat generation component can comprise iron powder, activated carbon, vermiculitm, and inorganic salt. In another embodiment, the heat generation component can comprise the heat generation component comprises a rare earth component. Such rare earth component can be a rare earth magnetic powder (e.g., samarium-cobalt magnetic material or neodymium based magnetic material) or a rare earth oxide, which can be any oxide of a rare earth element. Rare earth elements include lanthanide series elements and actinide series elements. Lanthanide series elements are Lanthanum, Cerium, Praseodymium, Neodymium, Promethium, Samarium, Europium, Gadolinium, Terbium, Dysprosium, Holmium, Erbium, Thulium, Ytterbium, and Lutetium; Actinide series elements are Actinium, Thorium, Protactinium, Uranium, Neptunium, Plutonium, Americium, Curium, Berkelium, Californium, Einsteinium, Fermium, Mendelevium, Nobelium, and Lawrencium.

As used herein, the term “a set of acupoints or one or more acupoint(s) for obesity” shall mean any set of acupoints or one or more acupoint(s) identified as acupuncture target for ameliorating or treating obesity. In some embodiments, the set of acupoints or one or more acupoint(s) shall include the set of acupoints comprises the shangwan, zhongwan, shenque, and Zusanli acupoints of a user (FIGS. 1A-1B).

### Acupuncture and Acupoints

Acupuncture, practiced for several thousand years in China (Veith I. The yellow emperor's classic of internal medicine. Williams & Wilkins: Baltimore; 1949. pp 58-76.), is increasingly used worldwide in the treatment of many disorders. An accumulating body of evidence summarized in a NIH Consensus Statement on Acupuncture (NIH Consensus Development Panel. Acupuncture. JAMA 1998; 280: 1518-1524) confirms that acupuncture treatment has beneficial effects for conditions ranging from postoperative dental pain to chemotherapy-associated emesis. It is also effective as an adjunctive modality for joint and muscle pain, addictions, and asthma, for example.

### Meridians and Acupoints

In Traditional Chinese Medicine (TCM), life force or ‘Qi’ (‘chee’) is thought to circulate within energy pathways or ‘meridians’ longitudinally throughout the body. There are 14 major meridians, corresponding (loosely) to the Western definition of ‘organs.’ Acupoints are specific locations on the body considered to be connected to these energy meridians (NIH Consensus Development Panel. Acupuncture. JAMA 1998; 280: 1518-1524; Vickers A, Zollman C. ABC of complementary medicine: acupuncture. BMJ 1999; 319: 973-976; and Mitchell E R. Fighting drug abuse with acupuncture: the treatment that works. Pacific View Press: Berkeley, Calif.; 1995). During illness Qi is thought to be out of balance, and stimulation of acupoints corrects this imbalance. Theoretically, an ‘excess’ or ‘deficiency’ of Qi can be ‘normalized’ by the specific manner of point stimulation.

In terms of traditional medicine, it is believed that acupuncture works to alter central nervous system neurotransmitter levels by stimulating peripheral nerves at acupoints. These stimulated nerves then carry the signals centrally (Stux G, Pomeranz B. Basics of acupuncture, 4th edn. Springer-Verlag: Berlin; 1998; Mann F. Acupuncture: cure of many diseases. Butterworth-Heinemann Ltd.: Oxford; 1992), shown in FIG. 3, including to the spinal cord, pituitary, and midbrain. Activated centers can then release neurochemicals: endorphins, monoamines, and cortisol (Stux G, Pomeranz B. Basics of acupuncture, 4th edn. Springer-Verlag: Berlin; 1998).

### Acupoint Stimulation

A large number of acupoints (365 points have been identified in Chinese acupuncture maps) show concentrations of peripheral nerve junctions, referred to as ‘trigger points (Vickers A, Zollman C. ABC of complementary medicine: acupuncture. BMJ 1999; 319: 973-976). A variety of methods are used to stimulate acupoints (NIH Consensus Development Panel. Acupuncture. JAMA 1998; 280: 1518-1524; Stux G, Pomeranz B. Basics of acupuncture, 4th edn. Springer-Verlag: Berlin; 1998; Lao L. Acupuncture technique and devices. J Altern Complem Med 1996; 2: 23-25). These include:
1. Needling or ‘traditional acupuncture’, in which fine stainless-steel needles are inserted through the skin to various depths, kept in place for varying lengths of time and/or further stimulated manually or electrically (‘electroacupuncture’).
2. Press needles or ‘staplepuncture’ in which short acupuncture needles are taped into place for extended periods with pressure being applied on a regular basis.
3. ‘Acupressure’ in which beads are massaged or pressed at specific locations.
4. ‘Moxibustion’ involving the application of heat, by burning small grain-sized pellets of combustible material on or near the acupoints.
5. ‘Cupping’ or using cups of various materials to create ‘negative pressure’ or a vacuum on the skin surface for increased blood circulation and point stimulation.
6. Transcutaneous electrical nerve stimulation (‘TENS’) which involves applying electrode pads to the skin surface which transmit a mild current into the acupoint.
   See, e.g., J M Lacey, et al., Acupuncture for the treatment of obesity: a review of the evidence, International Journal of Obesity (2003) 27, 419-427. doi:10.1038/sj.ijo.0802254.
   The teachings in the references in the preceding paragraphs are incorporated herein in their entirety by reference.

### Acupoints for Obesity

For providing treatment of obesity using device of invention, acupoints generally include shangwan, zhongwan, shenque, shenshu, taixi,and laogong. These acupoints are fully described in the acupoints diagram generally available in the art.

For a particular type of obesity, the acupoints include the above described set of acupoints and further, additional acupoints. Such additional sets of acupoints can be, for example, acupoints in the ears.

Treatment of obesity by acupuncture has been widely documented to be effective. See, e.g., J M Lacey, et al., Acupuncture for the treatment of obesity: a review of the evidence, International Journal of Obesity (2003) 27, 419-427; Cabýoglu M T, Ergene N, Tan U., The treatment of obesity by acupuncture, Int J Neurosci. 2006 February; 116(2):165-75. Beneficial effect of acupuncture includes, e.g., reduction of food or calorie intake, appetite, and/or improved mood in connection with diet control measures. Calorie intake can be reduced by, e.g., 25%, 50%, or about 30-40%.

For ameliorating or treating obesity of a particular patient, the acupoints include the above described set of acupoints and further, additional acupoints, which are generally known in the art.

### Formulations

The composition of invention can be formulated in any formulation suitable for topical, selective application to one or more acupoint(s) for obesity. One example of the formulation is a paste. Another example is a patch. The patch can include having a cover or protective layer one side of which has a composition of invention placed thereonto. The cover can be plastic, paper, or a foil (e.g., aluminum containing foil). A thin film can be used to sandwich the composition with the cover so as to prevent the composition from exposure to moisture prior to application to acupoint(s) for obesity of a user. A person of ordinary skill in the art can readily formulate a composition of invention into any of such formulations.

### EXAMPLES

### Example 1

### Complementary Treatment for Obesity

A composition disclosed herein is formulated into a patch and applied to one or any of the acupoints for obesity of a user. The patch generates heat to cause the temperature of the one or more of the acupoints to have a temperature of about 50° C., which lasts several hours. The procedure is repeated over a course of 3 months. Food intake is effectively reduced so as to result in weight loss.

While particular embodiments of the present invention have been shown and described, it will be obvious to those skilled in the art that changes and modifications can be made without departing from this invention in its broader aspects. Therefore, the claims are to encompass within their scope all such changes and modifications as fall within the true spirit and scope of this invention.

I claim:

1. A composition for obesity, comprising a heat generation component, wherein the composition is formulated in a formulation for selective application to a set of acupoints or one or more acupoint(s) for obesity (“acupoint(s) for obesity”) in a patient, wherein the composition generates heat upon application to the acupoint(s) for obesity for a sufficient period of time to generate an effect to ameliorate or treat obesity.

2. The composition of claim 1, wherein the set of acupoints comprises the shangwan, zhongwan, shenque, and Zusanli acupoints of a user.

3. The composition of claim 1 capable of generating heat for at least 1 hour after application to the set of acupoints or one or more acupoint(s) for obesity.

4. The composition of claim 1 capable of generating heat for at least 2 hours after application to the set of acupoints or one or more acupoint(s) for obesity.

5. The composition of claim 1, wherein the heat generation component comprises a rare earth component.

6. The composition of claim 5, wherein the heat generation component comprises a rare earth magnetic powder, saussurea in volucrata, musk, sea otter, chuanwu, caowu, lilac, menthol, meiyao, wujiapi, and maqianzhi.

7. The composition of claim 1, wherein the formulation is a patch comprising a cover.

8. The composition of claim 1, wherein the cover is a plastic, rubber, or paper cover.

9. A method of fabricating a composition for Obesity, comprising providing a heat generation component, and formulating the composition in a formulation for selective application to a set of acupoints or one or more acupoint(s) for obesity (“acupoint(s) for obesity”) in a patient, wherein the composition generates heat upon application to the set of acupoints or one or more acupoint(s) for obesity for a sufficient period of time to generate an effect to ameliorate or treat obesity.

10. The method of claim 9, wherein the composition is capable of generating heat for at least 1 hour after application to the set of acupoints or one or more acupoint(s) for obesity.

11. The method of claim 9, wherein the composition is capable of generating heat for at least 8 hours after application to the set of acupoints or one or more acupoint(s) for obesity.

12. The method of claim 9, wherein the heat generation component comprises a rare earth component.

13. The method of claim 12, wherein the heat generation component comprises a rare earth magnetic powder, saussurea in volucrata, musk, sea otter, chuanwu, caowu, lilac, menthol, meiyao, wujiapi, and maqianzhi.

14. The method of claim 9, wherein the formulation is a patch comprising a cover.

15. A method of ameliorating or treating obesity, comprising:
applying a composition at least once per day to a set of acupoints or one or more acupoint(s) for obesity (“acupoint(s) for obesity”) in a user, which composition comprising a heat generation component, wherein the composition is formulated in a formulation for selective application to the set of acupoints or one or more acupoint(s) for obesity over the entire length of a prescribed treatment course, wherein the composition generates heat upon application to the set of acupoints or one or more acupoint(s) for obesity for a sufficient period of time to generate an effect to ameliorate obesity.

16. The method of claim 15, wherein the composition is capable of generating heat for at least 1 hour after application to the set of acupoints or one or more acupoint(s) for obesity.

17. The method of claim 15, wherein the composition is capable of generating heat for at least 2 hours after application to the set of acupoints or one or more acupoint(s) for obesity.

18. The method of claim 15, wherein the heat generation component comprises a rare earth component.

19. The method of claim 18, wherein the heat generation component comprises a rare earth magnetic powder, saussurea in volucrata, musk, sea otter, chuanwu, caowu, lilac, menthol, meiyao, wujiapi, and maqianzhi.

20. The method of claim 15, wherein the formulation is a patch comprising a cover.

21. The method of claim 15, wherein the cover is a plastic, rubber, or paper cover.

22. The method of claim 15, wherein the treatment course lasts for about 1 month, 3 months, 6 months, or 12 months.
